(19) United States
(12) Patent Application Publication
(10) Pub. No.: US 2023/0348949 A1
(43) Pub. Date: Nov. 2, 2023

(54) MICROBIOLOGICAL PROCESS FOR THE PREPARATION OF URSOCHOLIC ACID

(71) Applicant: CHEMELECTIVA S.R.L., Novara (NO) (IT)

(72) Inventors: Michele CASTALDI, Novara (NO) (IT); Graziano CASTALDI, Briona (NO) (IT)

(21) Appl. No.: 18/016,904

(22) PCT Filed: Jul. 22, 2021
(86) PCT No.: PCT/EP2021/070506

§ 371 Date: Jan. 19, 2023

(30) Foreign Application Priority Data
Jul. 29, 2020 (IT) 102020000018397

(51) Int. Cl. C12P 33/06; C12N 1/14

(52) U.S. Cl. CPC C12P 33/06; C12N 1/145

(57) ABSTRACT

A microbiological process is provided for the preparation of ursocholic acid, which includes the biotransformation of β-sitosterol in the presence of specific microorganisms.

The present invention relates to a microbiological process for the preparation of ursocholic acid, starting from a sterol of vegetal origin.

Cholic acid is a primary bile acid synthesized in the liver from cholesterol through multiple complementary enzymatic processes. Bile acids include a group of molecular species with similar chemical structures, which are secreted into the bile and transported to the lumen of the small intestine where they act as emulsifiers promoting the digestion and absorption of fats, as well as by endocrine molecules capable of controlling different signaling routes.

The synthesis of bile acids represents the dominant metabolic pathway of cholesterol catabolism: 17 enzymes are involved in the production of these molecules. The final products defined as primary, such as cholic acid and chenodeoxycholic acid, can be modified by intestinal bacteria to form secondary bile acids such as ursodeoxycholic acid which, in turn, can be reabsorbed and returned to the liver via the enterohepatic circle. This is a strictly regulated synthesis to ensure that sufficient quantities of cholesterol are catabolized in order to maintain homeostasis and provide adequate emulsification in the intestine.

Contrary to the involvement of bile acids in the etiology and pathogenesis of various diseases, the physico-chemical and biological properties of these compounds have allowed them to be used in the development of drugs and pharmacological “tools” for the release of active agents.

The beneficial effects on health and the therapeutic use of bile acids were already known since ancient times. In particular, clinical studies have demonstrated their therapeutic efficacy, especially of ursodeoxycholic acid in the treatment of a broad spectrum of cholestatic liver diseases. During the twentieth century, only ursodeoxycholic acid and chenodeoxycholic acid were used. However, towards the end of the century, following the discovery of the ability of bile acids to activate the farnesoid X nuclear receptor (FXR), their effective therapeutic potential was recognized. In 2016, the US Food and Drug Administration (FDA) approved the use of cholic acid for congenital disorders of bile acid synthesis and as an additional remedy in peroxisomal disorders including Zellweger's cerebro-hepato-renal syndrome. In the same year, the use of obeticholic acid was approved, as a powerful selective agonist of FXR in the treatment of primary biliary cirrhosis in patients whose response to treatment with ursodeoxycholic acid was unsatisfactory.

In the last ten years numerous derivatives of bile acids have been synthesized and characterized; it is, in fact, a growing and promising field of biomedical research all over the world.

To date, the only economically viable resource of bile acids is bovine bile, which must be extracted at the time of slaughter. In slaughterhouses, the bovine gallbladder is recovered during meat processing and about 230 mL of bile can be obtained from a single cow, of which bile acids represent approximately 0.7% (w/w).

Only after extracting cholesterol, cholesterol esters, triglycerides and free fatty acids, the bile acids can be separated from the inorganic salts. To extract and purify the different bile acids, mainly primary bile acids, the bile is frozen and lyophilized: from 100 mL of bile 8 g of dry powder can be obtained. From these, about 6.9 g of 90% pure bile acids are obtained, from which, only later by means of a chemical transformation process, it will be possible to obtain colonic derivatives such as ursodeoxycholic acid (Beilstein J. Org. Chem. 2018, 14, 470-483).

This process entails considerable problems in terms of costs and environmental impact. One of the main problems concerns the availability of precursors for the synthesis of colic derivatives: the main producers of beef are located in newly industrialized countries, in particular in South America (Brazil) and India, where adequate technical conditions and hygienic protocols are often lacking, resulting in environmental pollution and the need to include sanitary procedures in the treatment of bile acids. Furthermore, in recent years, the raw material has undergone continuous and important price increases which have affected the finished product, which is becoming too expensive for pharmaceutical companies, companies which, in turn, are subject to price controls by organizations, health services of each country which, on the contrary, support policies to reduce them.

Hence the need to identify and develop an alternative source of transformable derivatives that allows to ensure high yields and competitive costs, reducing the complexity and the number of steps required for the synthesis, increasing the safety of the product.

The inventors of the present application have surprisingly found a method of preparation of ursocholic acid, an important precursor in the synthesis of bile acids whose chemical structure differs from that of cholic acid only by the inverse configuration of the hydroxy in position 7, which allows to overcome the drawbacks described above.

In fact, this method involves a single step and involves the biotransformation of a sterol of vegetal origin, β-sitosterol with a microorganism in a suitable culture broth. To date, no microbiological processes for the production of ursocholic acid seem to be reported.

Therefore, the subject of the present invention is a microbiological process for the preparation of ursocholic acid of formula (I)

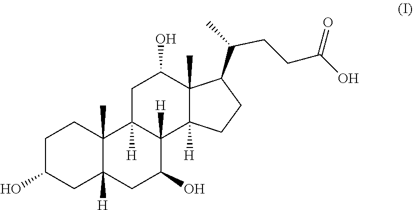

comprising the β-sitosterol biotransformation of formula (II)

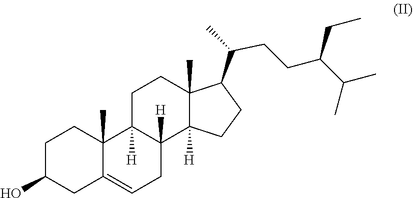

in the presence of a microorganism.

After extensive experimentation, the inventors of the present application have identified specific microorganisms that make it possible to obtain ursocholic acid with a high purity in a single step, i.e. more than 90%. Under particular experimental conditions which will be described later, the microorganism used in the process of the invention allows at the same time to reduce the double bond in position 5,6 of the β-sitosterol, to introduce the two hydroxyl groups in position 7β and 12α and to oxidize the branched alkyl chain to form the terminal carboxylic acid, without using long synthetic steps that would otherwise have been necessary with consequent low yields in order to obtain the desired product. The process according to the present invention is therefore highly regio- and stereoselective.

According to the invention, the microorganism suitable for the preparation of ursocholic acid is a fungal strain.

Preferably, said fungal strain is selected from Trametes spp., Botryosphaeria rhodina, Pleurotus ostreatus and Pleurotus incarnatus, more preferably it is selected from Trametes spp. wild type, Botryosphaeria rhodina DSM 62078, Botryosphaeria rhodina DSM 62079, Pleurotus ostreatus CBS 342.69, Pleurotus incarnatus CBS 498.76, even more preferably it is Pleurotus incarnatus CBS 498.76.

The preferred fungal strains according to the invention are identified with the filing number with organisms adhering to the Budapest Treaty, such as DSMZ (Deutsche Sammlung von Mikroorganismen and Zellkulturen) and CBS-KNAW.

These microorganisms can be used as lyophilisates or as a fresh culture isolated from the culture medium on which they are stored.

As is well known to the skilled person, in order to ensure proper growth of the microorganism and therefore the efficiency of the microbiological process, it is necessary to use an appropriate culture broth in specific temperature and pH conditions.

Examples of culture broths that can be used in the invention process are Donova, SAWADA and Bushnell-Haas Broth having the following compositions:
Donova: sucrose, KH₂PO₄, MgSO₄ x 7H₂O, Cornsteep liquor, H₂O;
SAWADA: oat flour, NaNO₃, KH₂PO₄, MgSO₄ x 7H₂O, FeSO₄ x 7H₂O, MnSO₄ x 7H₂O, H₂O;
Bushnell-Haas Broth: MnSO₄, CaCl₂, KH₂PO₄, K₂HPO₄, NH₄NO₃, FeCl₃

These culture broths are commercially available or can be prepared according to the methodology described in the Handbook of Media for Environmental Microbiology by Ronald M. Atlas, CRC Press (1995).

To bring the biotransformation of the invention to completion, the microorganism is present in the aforementioned culture broth in a concentration between 1 mg/l and 100 mg/l, preferably between 10 mg/l and 50 mg/l.

The microbiological process of the invention, or the preparation of ursocholic acid starting from a sterol of vegetal origin, is preferably carried out at a temperature of about 28° C. at a pH of about 7.0.

The biotransformation, object of the present invention, takes place by introducing a specific substrate into a fermenter containing the culture broth with the microorganism and leaving it to react for a determined period of time while maintaining the aforementioned temperature and pH constant.

The substrate of the present invention is a sterol of vegetal origin, β-sitosterol, present in vegetable oils, such as for example soybean oil, rapeseed oil, corn oil, but also in nuts or avocados.

This sterol has numerous advantages, including its availability on the market and the low cost corresponding to 10 times less than the current cost of cholic acid obtained by extraction.

The β-sitosterol can be incorporated into the culture broth as such or suspended in water.

Preferably the substrate concentration is between 1 and 10 g/l, more preferably it is about 2 g/l.

Furthermore, according to a preferred embodiment of the invention, the reaction time required to transform β-sitosterol into ursocholic acid by the action of the microorganism is between 120 and 360 hours, more preferably it is about 240 hours. According to a particularly preferred embodiment, the strain is left to grow in the absence of substrate in the presence of the broth at a temperature of 28° C. and a pH of about 7.0 for about 72 hours, after which the β-sitosterol is introduced into the fermenter to a final concentration of approximately 2 g/l in which the Donova culture broth contains the P. incarnatus strain. It is left under stirring for about 240 hours at a temperature of 28° C. and a pH of about 7.0 to form ursocholic acid.

Once the reaction is over, the isolation and purification of the ursocholic acid thus obtained are carried out by techniques well known to those skilled in the art, such as centrifugation of the culture broth, separation of the solid, adjustment of the pH of the solution to 2-3. with acetic acid and extraction with ethyl acetate. The organic phase is washed with water and evaporated to give the desired product with a high purity of about 90%. If necessary, the product can be further purified, for example by crystallization, according to the techniques known in the art.

As mentioned above, ursocholic acid is an important precursor in the synthesis of bile acids.

The ursocholic acid thus obtained can then be subsequently subjected to further chemical processes aimed at obtaining cholic derivatives such as ursodeoxycholic acid (III), chenodeoxycholic acid (IV), obeticholic acid (V), lithocholic acid (VI) or cholic acid (VII), whose chemical structures are shown in the diagram below.

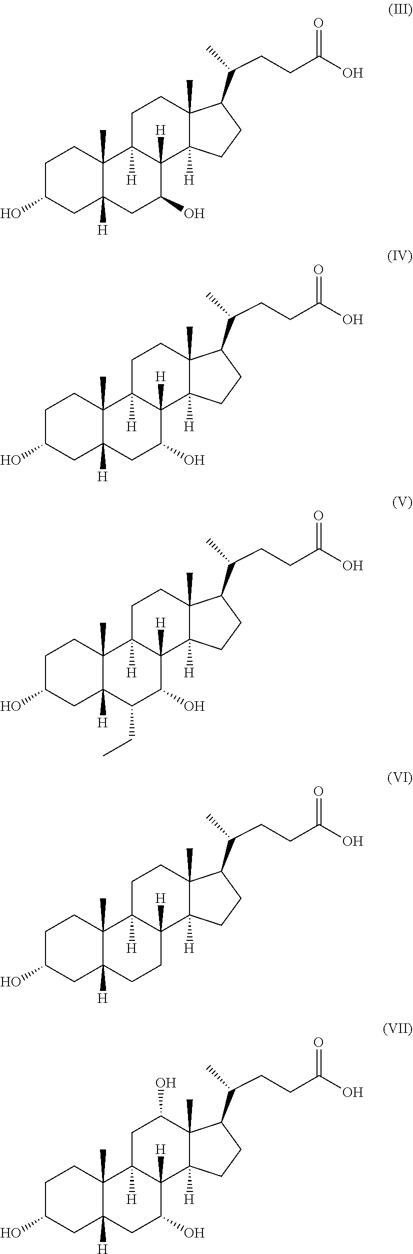

For example, ursodeoxycholic acid (III) can be prepared starting from ursocholic acid of formula (I) following the procedure described in patent EP 72 293.

As for the other cholic derivatives (IV), (V), (VI) and (VII) they can be obtained using synthetic methods well known to the skilled person.

The conversion of ursocholic acid obtained according to the process of the present invention into a cholic derivative as reported above represents a further object of the invention.

The present invention will now be illustrated by means of some examples, which should not be seen as limiting the scope of the invention.

### EXAMPLES

### Example 1

5.0 g of magnesium sulfate, 5.0 g of monopotassium phosphate, 50.0 g of sucrose, 20.0 mL of Cornsteep liquor and water are loaded into a 2 L fermenter together with the Pleorotus incarnatus strain. It is left under stirring for 72 hours.

2 g of β-sitosterol are subsequently added to the mixture and it is left under stirring (160 rpm) for 240 hours at 28° C.

At the end the mass is isolated and purified by centrifugation of the culture broth. The solid is separated from the liquid phase, the pH of the solution is adjusted to about 2-3 with acetic acid and extracted with ethyl acetate. The organic phase is washed with water and evaporated to give 1.8 g of ursocholic acid with HPLC purity of 90%.

mp 145-146° C.; [α] D +68.8° (c 0.5, EtOH); 1H-NMR δ 3.80 (1 H, t, J=2.8 Hz, H-12β), 3.33 (2 H, m, H-3βand H-7α), 0.94 (3 H, d, J=7 Hz, C-21 Me), 0.86 (3 H, s, C-19 Me), 0.63 (3 H, s, C-18 Me).

### Example 2

5.0 g of magnesium sulfate, 5.0 g of monopotassium phosphate, 50.0 g of sucrose, 20.0 mL of Cornsteep liquor and water are loaded into a 2 L fermenter together with the Pleorotus incarnatus strain. It is left under stirring for 72 hours.

2 g of β-sitosterol are subsequently added to the mixture and it is left under stirring (160 rpm) for 240 hours at 28° C.

At the end the mass is isolated and purified by centrifugation of the culture broth. The solid is separated from the liquid phase, the pH of the solution is adjusted to about 2-3 with acetic acid and extracted with ethyl acetate. The organic phase is washed with water and evaporated to give 1.8 g of ursocholic acid with HPLC purity of 90%.

mp 145-146° C.; [α] D+68.8° (c 0.5, EtOH); 1H-NMR δ 3.80 (1 H, t, J=2.8 Hz, H-12β), 3.33 (2 H, m, H-3β and H-7α), 0.94 (3 H, d, J=7 Hz, C-21 Me), 0.86 (3 H, s, C-19 Me), 0.63 (3 H, s, C-18 Me).

1. A microbiological process for the preparation of ursocholic acid of formula (I) 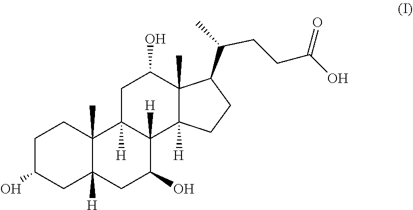
comprising the biotransformation of β-sitosterol of formula (II) 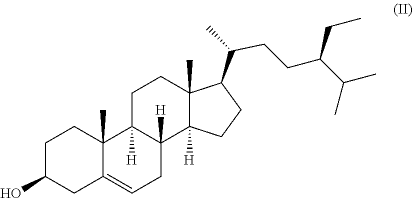
in the presence of Pleurotus incarnatus.

2. The process according to claim 1, wherein Pleurotus incarnatus is Pleurotus incarnatus CBS 498.76.

3. The process according to claim 1, wherein the biotransformation occurs in an adequate culture broth.

4. The process according to claim 1, wherein the Pleurotus incarnatus concentration is comprised between 1 mg/l and 100 mg/l.

5. The process according to claim 1, wherein the β-sitosterol concentration is comprised between 1 g/l and 10 g/l.

6. The process according to claim 1, wherein the biotransformation occurs in a time comprised between 120 hours and 360 hours.

7. A process for the preparation of a cholic derivative comprising the process for the preparation of ursocholic acid according to claim 1.

8. The process for the preparation of a cholic derivative according to claim 7, wherein the cholic derivative is selected from ursodeoxycholic acid, chenodeoxycholic acid, obeticholic acid, lithocholic acid and cholic acid.

9. The process according to claim 6, wherein the biotransformation occurs in a time of about 240 hours.

10. The process according to claim 5, wherein the β-sitosterol concentration is about 2 g/l.

11. The process according to claim 4, wherein the Pleurotus incarnatus concentration is comprised between 10 mg/l and 50 mg/l.

12. The process according to claim 3, wherein the culture broth is selected from Donova, SAWADA, and Bushnell-Haas Broth.

13. The process according to claim 2, wherein the biotransformation occurs in an adequate culture broth.

14. The process according to claim 13, wherein the culture broth is selected from Donova, SAWADA, and Bushnell-Haas Broth.

15. The process according to claim 13, wherein a concentration of Pleurotus incarnatus is between 1 mg/l and 100 mg/l.

16. The process according to claim 13, wherein the Pleurotus incarnatus concentration is comprised between 10 mg/l and 50 mg/l.

17. The process according to claim 15, wherein a concentration of β-sitosterol is between 1 g/l and 10 g/l.

18. The process according to claim 17, wherein the β-sitosterol concentration is about 2 g/l.

19. The process according to claim 17, wherein the biotransformation occurs in a time comprised between 120 hours and 360 hours.

20. The process according to claim 19, wherein the biotransformation occurs in a time of about 240 hours.
